(19) United States
(12) Patent Application Publication
(10) Pub. No.: US 2005/0054965 A1
(43) Pub. Date: Mar. 10, 2005

(54) OVRsite cover

(76) Inventor: E. Jacquelyn Kirkis, Tucson, AZ (US)

Correspondence Address: E. Jacquelyn Kirkis, 9041 E. Kenyon Drive, Tucson, AZ, 85710, US

(21) Appl. No.: 10/654,310

(22) Filed: Sep. 4, 2003

(51) Int. Cl. A61F 13/00

(52) U.S. Cl. 602/42

(57) ABSTRACT

A domed, cup shaped, clear, non-crushable, vented with holes or openings, to place over the healing pustule of the vaccination site for smallpox, is provided as a protective dressing to prevent touch by clothing or hands, and to restrict the risk of dissemination of the viral particles from the moist healing pustule, to scatter or aerosolize from the vaccination site. The cover is formed of non-crushable plastic with 2 side projections (phalanges) to be used to tape the dome securely to the skin. The cover is single use, disposable, environmentally safe, and economical. The cover is clear allowing daily inspection of the healing site without removal of the dressing, thus disturbing the healing process. The cover is non-crushable preventing clothing, hands or any other source touch the the site of the vaccination. The cover allows safe bathing or showering.

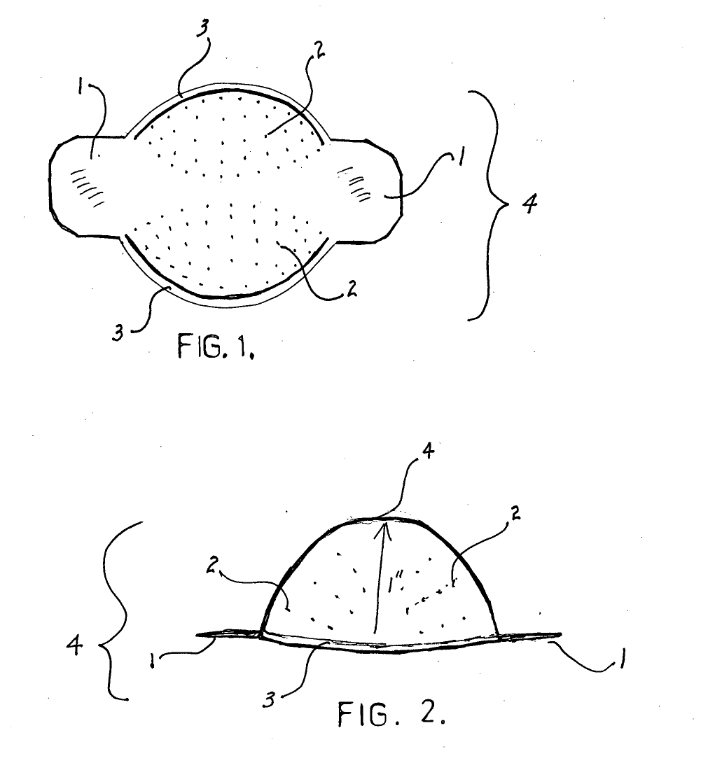

### BACKGROUND OF INVENTION

This invention relates to a cover to protect the forming wet pustule resulting from the administration of the vaccination for smallpox.

More particularly, this cover relates to the protection as being a non-crushable clear, vented cover, with holes or openings to allow the circulation of air to assist in drying the wet surface of the vaccination pustule; and secondly the protection of the site from touch by clothing, hands, or other mechanisms which could harm the healing area, yet allow the observation of the healing process without removing the protective cover; and thirdly a prevention method against the dissemination of the viral particles from the surface of the healing pustule.

Many types of bandages, and covers are available for the protection of wounds. Wound wrappings of all types used as protection and to assist healing, have included moss, leaves, skins, cloth rags, coming to the formal sterilized gauze pads, cellulose packs, absorbent drainage pads, and the use of the synthetic polymer covers of the 21^{st} century which are now provided by commercialized vendors.

The smallpox vaccination site has been covered traditionally with gauze pads, to prevent the touch by clothing or hands during the healing period. The marketing of the clear self adhering covers called Op-Cite™ or Tegaderm™ and others of similar synthetic polymers has been directed toward use over the smallpox vaccination site, as a dressing of choice.

Authorities such as the Centers for Disease and Prevention, Atlanta, Ga. (CDC) and the Advisory Committee on Immunization Practice (ACIP) have published their recommendations for the type of dressing or cover to use over the healing site. In these recommendations the traditional approach is maintained as gauze which is porous, to be removed daily for inspections, and the site is to be covered until the healed scab falls off the site. Gauze adheres to the wet and drying surface of the pustule which is pulled off each time the gauze dressing is removed, which would be daily. With such a dressing, the viral particles which are in the wet drainage, and on the surface of the open pustule, are at risk for becoming air borne into the immediate area, raising the risk of exposure to others. Some who have used the traditional dressings have had no problem with the forming scab being pulled off with the removal of the dressing. Others however have had the scab pulled off and the healing process continually retarded with daily removal of the cover.

In the decades of the 1920s until 1977, when smallpox vaccinations were routine, a cover similar to the OVRsite COVER™ was marketed with great success and very little problem with protection of the vaccination site. This cover had no patent nor copyright The OVR site COVER™ is a redesign of this effective protection for the smallpox vaccination site. The urgency of preparing for terrorists attacks using biologicals such as smallpox against a population, requires that all factors be considered, including the need for a simple, and effective, convenient, economical approach to handling vaccinated civilians and military. There will be civilians caring for civilians. Changing of dressings on smallpox vaccination sites, daily, will create a burden for the health teams, a burden which will be delegated to those civilians. The use of a single dressing which can be kept in place, yet allows for the inspection of the site without removal of the dressing, and protects the site from touch even in the bath or shower, will relieve the untrained care giver or over seer who will have the responsibility. Such a dressing will be practical and desired.

### BACKGROUND & STATEMENT OF FEDERAL SPONSORED R&D

It is the principle objective of the present invention, OVRsite COVER™, to provide the practical dressing which will meet the criteria of the oversight agencies with porosity, no touch, visual observation, protection of the vaccination site.

The present invention is recognized to be subject to many changes and modifications, without departing from the spirit or essential characteristics of the present invention, OVRsite COVER™, as set forth in the appended claim.

### STATEMENT OF FEDERAL SPONSORED R & D

There has been no sponsorship of the research and development of this cover. All research and development has been by the principle inventor who is a licensed healthcare provider with over 58 years of healthcare experience here in the United States and abroad, as a practitioner, professor, and administrator.

### SUMMARY OF THE INVENTION

In accordance with the foreging background description the invention, OVRsite COVER™, provides a unique protecive dressing cover for the smallpox vaccination site. The cover is formed from non-crushable plastic in the shape of a small dome. The dome, with phalanges on each side, provides a method for which tape can secure the cover to protect the healing site of the vaccination, and the rising pustule. The dome is vented with holes or openings to allow air circulation. The dome is clear allowing for visual inspection of the pustule site. The dome does not touch the healing pustule.

The domed, clear, non-crushable dressing is sized for adult, child, or infant. The domed, clear, non-crushable dressing can be kept in place during bath or shower. In some vaccinations there may be excessive exudate from the pustule, as the scab is forming. The domed, clear, non-crushable dressing can be lifted at one side by releasing the tape over the phalange. The exudate is swabbed away by using sterile swabs, or sterile pads. The domed cover is retaped in place, and healing of the scab is undisturbed.

### HEALTH PROCEDURES, DRESSINGS, WOUND COVERS

Patent search October 2002, through March 2003 for time period of 1920 through 1977 revealed no such protective cover or dressing had received a patent now was under a patent.

There are many wound dressings marketed under company trademarks. None of dressings or bandages or covers for wounds, or vaccinations are similar nor do any resemble this cover. None of the marketed dressings or wound covers are specific for the cover or the protection of the smallpox vaccination site.

1. A non-crushable, clear, dome shaped dressing which is vented with holes or openings which is placed over the healing pustule which results from a smallpox vaccination.
a. The cover, fashioned of non-crushable plastic in the shape of a dome, or cup, with two side projections or phalanges which lie flat on the skin, over which tape is used to secure the dome in place over the healing vaccination site. The dome does not touch the healing site.
b. The cup or dome is vented with holes or openings in a regular pattern over the top of the dome. This allows air circulation for healing of the vaccination pustule to form the scar of indication.
c. The dome is fashioned to rise over the pustule without touching the surface of the pustule. The height and diameter are formed in sizes to accommodate adult or child vaccinations.
d. The domed, clear, vented, non-crushable cover is designed to be a one time use by a single individual. The cover is disposable.
e. The design of the cup or domed, vented, clear, cover can be lifted by releasing the tape over one of the side projections, in order to swab or cleanse excess exudate from the healing pustule: then re-tape the side phalange (projection.)

2. The domed, clear, non-crushable, vented cover, dependant on claim 1 defines the healing pustule of the vaccination to dry scab, as the area for protection.

3. Formed of non-crushable plastic, vented, clear and shaped to prevent touch of the healing vaccination site, dependant on claim 1, providing protection during bathing or showering.

4. The domed, clear, vented, non-crushable cover, dependant on claim 1, is not designed as “left” or “right” therefore will fit over any vaccination site.

5. The domed, vented, clear, non-crushable cover, dependant on claim 1, is formed as a single cup with 2 side projections (phalanges) to allow tape to secure the cover to the skin.

6. The domed, vented, clear, non-crushable cover, dependant on claim 1, is formed of environmentally safe material, allowing for disposal in the regular trash, or landfill.
